(19) United States
(12) Patent Application Publication
(10) Pub. No.: US 2026/0049919 A1
(43) Pub. Date: Feb. 19, 2026

(54) REAL TIME MONITORING OF EXPOSURE ATMOSPHERE AT MULTIPLE LOCATIONS IN AN AEROSOL EXPOSURE SYSTEM USING A SINGLE REALTIME MONITOR

(71) Applicant: Battelle Memorial Institute, Columbus, OH (US)

(72) Inventors: Amit GUPTA, Dublin, OH (US); Janae NEWSWANGER, Galloway, OH (US); Cassady GRIFFIN, Columbus, OH (US)

(21) Appl. No.: 19/287,998

(22) Filed: Aug. 1, 2025

Related U.S. Application Data
(60) Provisional application No. 63/683,799, filed on Aug. 16, 2024.

(51) Int. Cl. G01N 15/06; G01N 1/22; G01N 1/38; G01N 15/00; G01N 33/00

(52) U.S. Cl. CPC G01N 15/06; G01N 1/2202; G01N 1/38; G01N 2001/2223; G01N 2015/0046; G01N 2015/0681; G01N 33/0098

(57) ABSTRACT

In an approach to real time monitoring of exposure atmosphere in an aerosol exposure system, a system includes one or more aerosol inputs; a mixing bulb; a dilution manifold with a plurality of dosimetry positions; a multiport valve; and a real-time aerosol monitor (RAM). The system is configured to: receive aerosol samples from the one or more aerosol inputs; mix the aerosol samples with humidified air using the mixing bulb; input the humidified aerosol samples into the dilution manifold; for each dosimetry position of the plurality of dosimetry positions: measure an exposure concentration for the humidified aerosol sample from the dosimetry position of the dilution manifold using the RAM; and select a next dosimetry position of the dilution manifold.

### CROSS-REFERENCE TO RELATED APPLICATIONS

The present application claims the benefit of the filing date of U.S. Provisional Application Ser. No. 63/683,799, filed Aug. 16, 2024, the entire teachings of which application is hereby incorporated herein by reference.

### TECHNICAL FIELD

The present disclosure relates generally to toxicology and, more particularly, to a system and method for real time monitoring of exposure atmosphere at multiple locations in an aerosol exposure system using a single real time monitor.

### BACKGROUND

Continuous development and implementation of in vitro test methods are driven by the need for faster and more human-relevant health risk assessment across toxicology communities, including research on tobacco and nicotine products. For standard in vitro testing of inhalable products, test materials are exposed to multiple concentrations of aerosol in a single experiment. Real time monitoring of aerosol concentrations in all dose groups is critical to ensure the test material are exposed to correct quantities of aerosol and maintain quality of data during the study execution (e.g. maintain consistent aerosol atmosphere and reduce variability over time).

### BRIEF DESCRIPTION OF THE DRAWINGS

Reference should be made to the following detailed description which should be read in conjunction with the following figures, wherein like numerals represent like parts.

FIG. 1 is a functional block diagram illustrating a system for real time monitoring of exposure atmosphere in an aerosol exposure system consistent with the present disclosure.

FIG. 2 is a chart of temporal variability of the aerosol concentration in the mixing bulb for one example of the system of FIG. 1.

FIG. 3 is a table-1 of port equivalency measurements for the experiment of FIG. 1.

FIG. 4 is a table-2 of the measured RAM response and analytical measurements compared to theoretical estimates for the experiment of FIG. 1.

FIG. 5 is an example of a software architecture for a system and method for real time monitoring of exposure atmosphere in an aerosol exposure system consistent with the present disclosure.

FIG. 6 is an example of a method for real time monitoring of exposure atmosphere in an aerosol exposure system consistent with the present disclosure.

FIG. 7 is a flowchart diagram depicting operations for the method for real time monitoring of exposure atmosphere in an aerosol exposure system, consistent with the present disclosure.

### DETAILED DESCRIPTION

Disclosed herein is a system and method for real time monitoring to assess exposure atmosphere in an aerosol exposure system. The disclosed system provides for almost real time automatic assessment of test atmosphere concentration. While existing systems require multiple real-time aerosol monitors (RAMs) for real time monitoring of multiple dosimetry positions, the disclosed system uses a multiport valve to select a dosimetry position to monitor, allowing for monitoring of multiple dosimetry positions using only a single monitor. Since only one monitor is used, the disclosed system provides more efficiency in time and costs (i.e., less number of real time monitors to install and maintain).

As disclosed herein, a fit-for-purpose characterization of an air liquid interface (ALI) exposure system was conducted using whole aerosol produced from a commercial heated tobacco product. Three goals were set for this characterization. First, producing continuous and stable aerosols from a commercial heated tobacco product (HTP), second, widening the range of target concentrations that can be tested in a single experiment, and third, selective characterization of aerosols in the exposure system, including real-time monitoring of aerosol concentrations, aerosol particle size measurements and analytical measurements of nicotine and glycerol collected in liquid solvent traps.

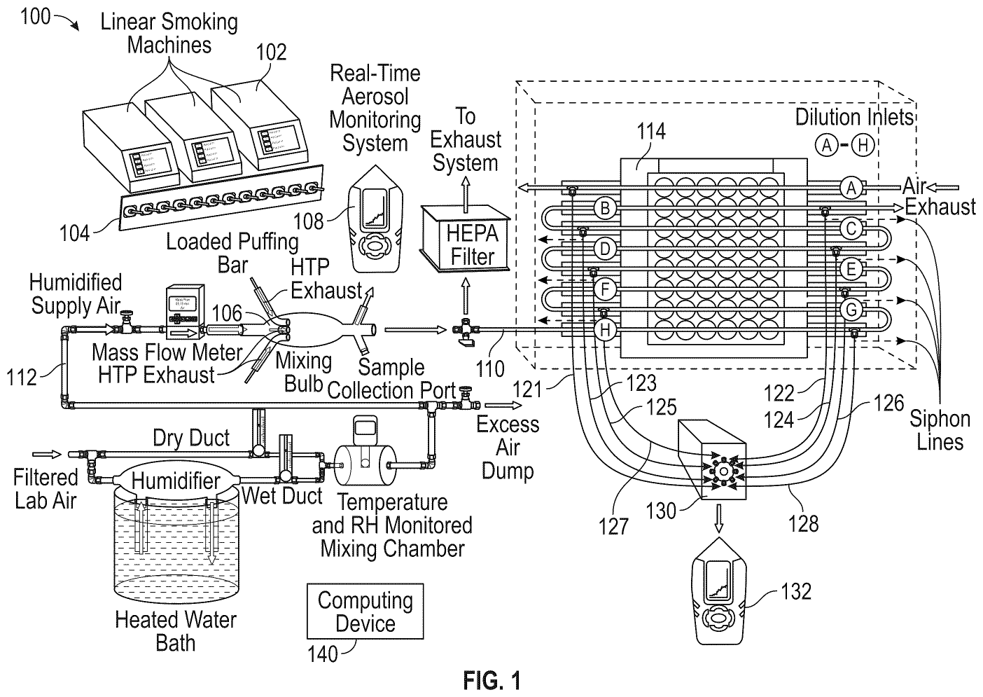

FIG. 1 is a functional block diagram illustrating one example of a system 100 for real time monitoring of exposure atmosphere in an aerosol exposure system consistent with the present disclosure. It should be noted that the example system 100 of FIG. 1 described herein may be capable of other embodiments and of being practiced or being carried out in various ways.

The example system 100 includes one or more linear smoking machines 102 connected to a loaded puffing bar 104. Output from the one or more linear smoking machines 102 are mixed with a humidified air from humidified air supply 112 by a custom mixing bulb 106 to ensure the dilution airflow is at a predetermined desired relative humidity (RH). The output of the mixing bulb 106 may be sampled by a RAM 108 to measure the temporal variability of aerosol concentrations in the mixing bulb. The output of the mixing bulb 106 is then introduced into a dilution manifold 114 to analyze the aerosol samples and quantify the concentration.

The dilution manifold 114 may allow for analysis of the input samples at different dilution levels. In the example of FIG. 1, the dilution manifold 114 is configured to allow sampling at seven different dilution levels, but in other embodiments, dilution manifolds with other numbers of different dilution levels may be used. The dilution manifold 114 includes eight dosimetry positions, dosimetry position-1 121 through dosimetry position-8 128. In this example, dosimetry position-8 128, closest to the input from the mixing bulb 103, has the highest concentration of the aerosol, while the dosimetry position-2 122, furthest from the input from the mixing bulb 103, has the lowest concentration of the aerosol (dosimetry position-1 121 in this example is a filtered air control position).

A multiport rotation valve 130 allows for sampling of any of the dosimetry position-1 121 through dosimetry position-8 128. The input to the multiport rotation valve 130 is from the dosimetry position-1 121 through dosimetry position-8 128, and the output of the multiport rotation valve 130 is the input to a second real-time aerosol monitor 132.

The system 100 also includes computing device 140. Computing device 140 can be a standalone computing device, a management server, a web server, a mobile computing device, or any other electronic device or computing system capable of receiving, sending, and processing data. In an embodiment, computing device 140 can be a personal computer, a laptop computer, a tablet computer, a netbook computer, a smart phone, or any programmable electronic device capable of communicating with other computing devices (not shown) within system 100. In another embodiment, computing device 140 can represent a server computing system utilizing multiple computers as a server system, such as in a cloud computing environment. In yet another embodiment, computing device 140 represents a computing system utilizing clustered computers and components (e.g., database server computers, application server computers) that act as a single pool of seamless resources when accessed within distributed data processing environment 100.

The following is a description of one example experiment performed using the system 100 of FIG. 1. In the example experiment, a modified ISO 20778 puffing regimen (55 mL puff volume, 2 s puff duration, 30 s puff interval, bell-shaped puff profile, no vent blocking) was used to produce aerosols from a commercially available HTP product. An optimized staggered loading pattern was developed to load twelve HTPs on a linear smoking machine (for example, an LM24E from Koerber Instruments GmbH, Hamburg, Germany). As shown in FIG. 1, the custom-designed mixing bulb 106 installed at the output of the smoking machine allowed proper mixing of the aerosols by introducing a predetermined humidified dilution air flow (for example, a target relative humidity RH of approximately 80%). The real-time aerosol monitor 108 (for example, a Casella MD Pro manufactured by TSI Incorporated of Shoreview, Minnesota,) was used to measure the temporal variability of aerosol concentrations in the mixing bulb.

Mixed aerosol was then introduced into an exposure system (for example, a VITROCELL® 24/48 ALI manufactured by VITROCELL Systems GmbH, Waldkirch, Germany). By using the dilution manifold 114 with a trumpet inlet flow set to 2 mL/min, seven target concentrations were achieved (dosimetry position-2 122 through dosimetry position-8 128), in addition to a filtered air control position (dosimetry position-1 121). Siphons were connected at the end of each row to widen the range of exposure concentrations, with, for this example, a low concentration targeted as ˜2% of the highest concentration that can be tested in a single experiment. A second RAM 132 was installed to monitor the stability of the aerosol concentrations at each concentration group. An automatic multiport rotation valve 130 was installed at the inlet of the RAM 132 that enabled monitoring of multiple locations using a single RAM 132. Aerosols were collected during the exposure in one well containing a liquid solvent trap (for example, phosphate buffered saline (PBS), available from Sigma Aldrich) for each concentration group and quantified for nicotine and glycerol. Aerosol particle size was measured in each row using a Mercer cascade impactor (for example, a Model M manufactured by In-Tox Products of Clinton, MS) and an optical particle sizer (for example, a Model 3330 manufactured by TSI Inc, Shoreview, MN).

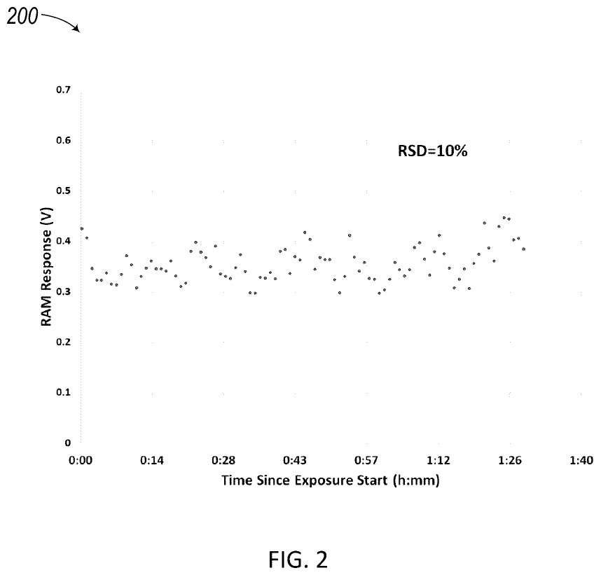

FIGS. 2-4 contain the results of the experiment described under FIG. 1 above.

FIG. 2 is a chart 200 of temporal variability of the aerosol concentration in the mixing bulb for the experiment of FIG. 1. The results of the experiment of FIG. 1 demonstrate that aerosol concentration in the mixing bulb was temporally stable; the relative standard deviation (RSD) of aerosol concentration as shown in the figure was 10% (i.e., a relative standard deviation measurement of less than 15% is considered acceptable).

FIG. 3 is a table-1 300 of port equivalency measurements for the experiment of FIG. 1. During the setup phase, port equivalency measurements were collected. The grand RSD 302 of all measurements was 12.3% and the percent difference of grand mean from mean 304 of each valve position was less than or equal to 8%. These measurements showed no bias associated with the multiport rotation valve.

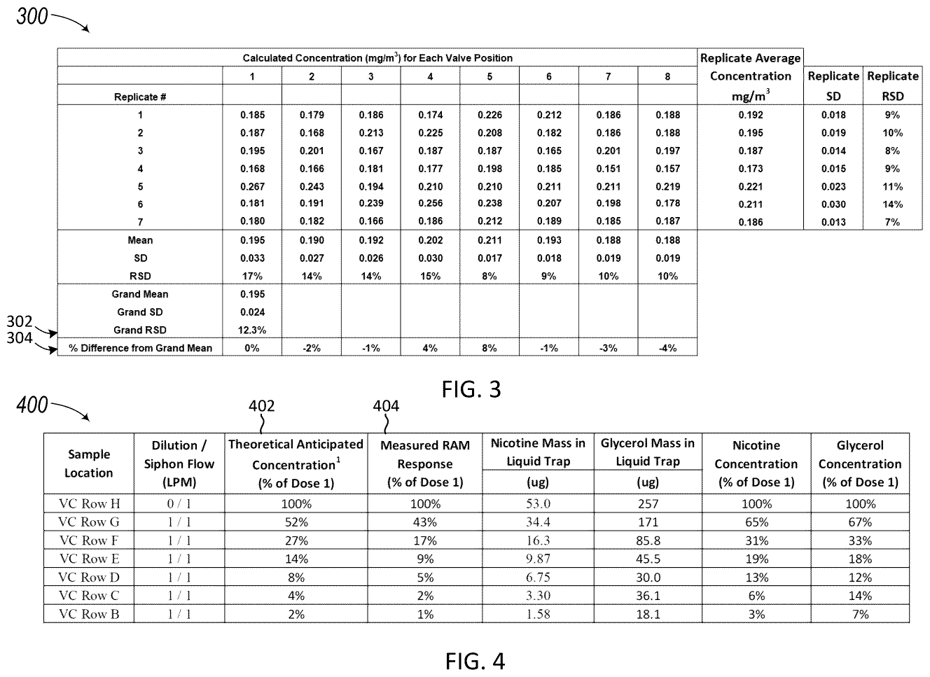

FIG. 4 is a table-2 400 of the measured RAM response and analytical measurements compared to theoretical estimates for the experiment of FIG. 1. It should be noted that the Theoretical Anticipated Concentrations 402 shown in column-3 are theoretical calculations based on combination of dilution and siphon flows in each row. Since trends with measured RAM responses 404 were consistent with those of theoretical anticipated concentrations 402, it further demonstrated there were no significant losses, or biases, introduced by the multiport valve used at the inlet of RAM.

The evaluation experiments concluded that fit-for-purpose methods were developed to generate stable and consistent aerosols from a commercial HTP product in an ALI exposure system. A well-controlled, wide dynamic range of exposure concentrations monitored in real-time using a single RAM were achieved. This allows designing dose range finding studies with a wide range of exposure concentrations and more precise monitoring and control of aerosol concentrations during the exposures.

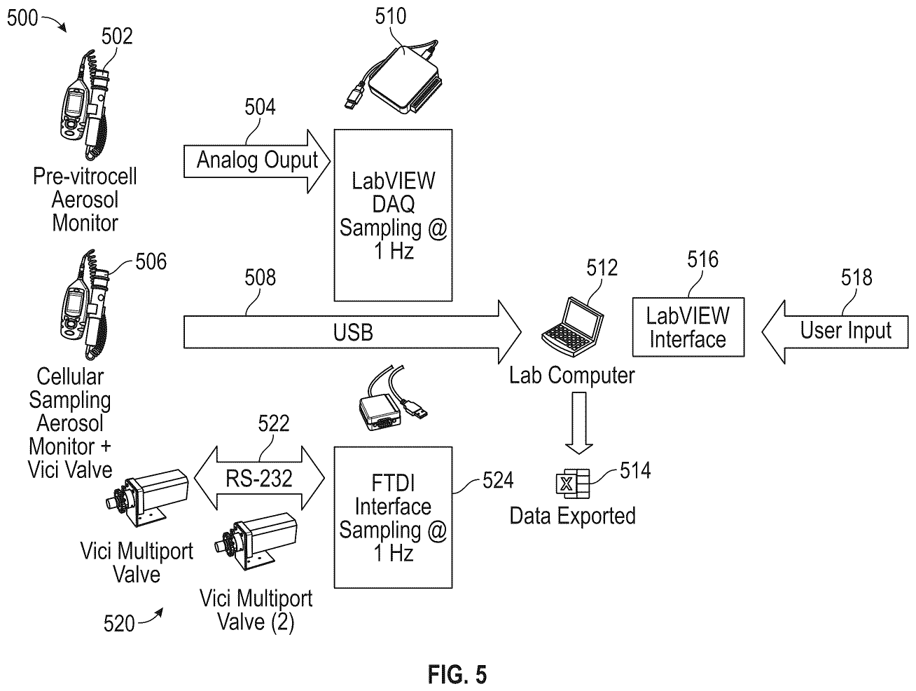

FIG. 5 is an example of a software architecture 500 for a system and method for real time monitoring of exposure atmosphere in an aerosol exposure system consistent with the present disclosure. The software architecture 500 includes a pre-analysis RAM 502 to sample the aerosol prior to the aerosol exposure system. The output 504, which may be an analog output, of the RAM 502 is sent to the aerosol exposure system, which may be sampled by a laboratory data acquisition system 510, for example, a LabVIEW DAQ made by the National Instruments division of Emerson. A cellular sampling aerosol exposure system 506 may monitor the data from the aerosol exposure system, and the results may be sent to a user interface, e.g., a graphical user interface (GUI), such as displayed on lab computer 512. The GUI may be, for example, the LabVIEW interface 516, which is controlled by a user through user input 518. The lab computer 512 may also export the data to, for example, a spreadsheet 514.

The architecture 500 may include one or more multiport valves 520 which may be, for example, multiport rotation valve 130 from FIG. 1, to select the dosimetry position from which the sample is extracted. The one or more multiport valves 520 may communicate with the laboratory data acquisition system 510 over a bus, such as an RS-232 bus 522, or via an FTDI interface 524. The one or more multiport valves 520 may be controlled by the user through the lab computer 512 or may be configured to automatically select the dosimetry position at a predetermined rate. For example, the one or more multiport valves 520 may be configured to step through the dosimetry positions sequentially at a 1 Hz rate.

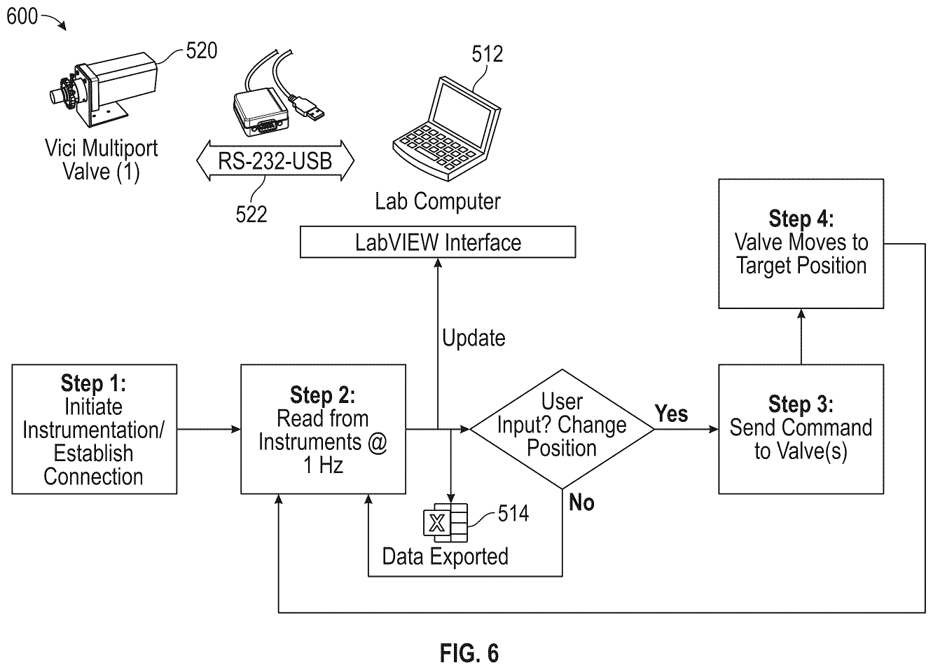

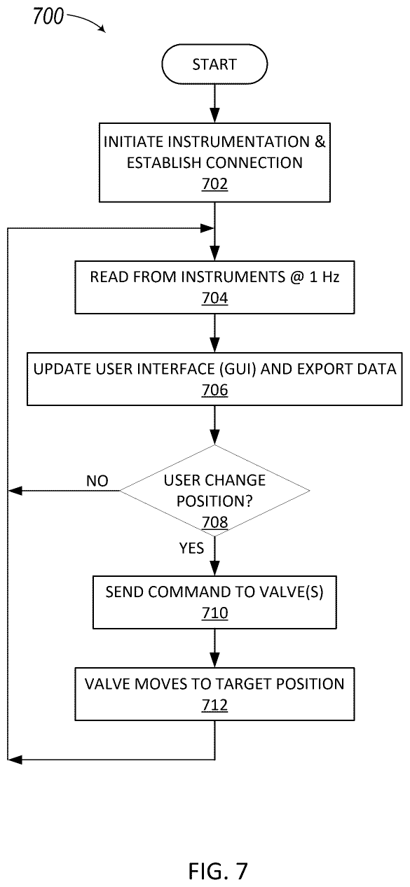

FIG. 6 is another example of a method 600 for real time monitoring of exposure atmosphere in an aerosol exposure system consistent with the present disclosure. The method 600 of FIG. 6 is explained in the flowchart of FIG. 7 below.

FIG. 7 is a flowchart diagram depicting operations for the method 700 for real time monitoring of exposure atmosphere in an aerosol exposure system, consistent with the present disclosure. It should be appreciated that FIG. 7 provides only an illustration of one implementation and does not imply any limitations with regard to the environments in which different embodiments may be implemented. Many modifications to the depicted environment may be made by those skilled in the art without departing from the scope of the disclosure as recited by the claims.

Method 700 includes initiating instrumentation and establish connection (operation 702). In the illustrated example embodiment, the system initializes the instrumentation and establishes connection to the instrumentation. In an embodiment, the system may initialize the instrumentation by establishing communications with the instrumentation and sending an initialization signal to the instrumentation. In another embodiment, the system may initialize the instrumentation by remotely powering the instrumentation on and connecting it to the instrumentation once it has powered up. In other embodiments, the system may initialize the instrumentation and establish a connection to the instrumentation by any other appropriate means, as would be known to one skilled in the art.

Method 700 includes reading from instruments at a 1 Hz rate (operation 704). In operation 704, data is read from the instruments at a predetermined rate. In an embodiment, the predetermined rate may be 1 Hz. In other embodiments, any other appropriate sample rate may be used as would be known to one skilled in the art.

Method 700 includes updating user interface (GUI) and export data (operation 706). In operation 706, for each cycle of operation 704, the data read from the instruments is used to update a user interface on a user's computer. In an embodiment, the user interface may be a GUI. In an embodiment, the data from the instruments may be exported for offline analysis, for example, to a spreadsheet or database.

Method 700 includes determining whether the user provided an input to change the position (decision block 708). If the system determines that the user did provide an input to change the dosimetry position (“yes” branch, decision block 708), then the system proceeds to operation 710. If the system determines that the user did not provide an input to change the dosimetry position (“no” branch, decision block 708), then the system returns to operation 704 to continue to read data from the instruments.

Method 700 includes sending a command to the valve(s) (operation 710). If the system determines in decision block 708 that a user input was received to change the dosimetry position, then in operation 706 the system will send a signal to the multiport rotation valve instructing the valve to rotate to the desired target position.

Method 700 includes moving the valve(s) to the target position (operation 712). Once the signal is sent to the multiport rotation valve instructing the valve to rotate to the desired target position, the valve responds by rotating to the target position. The system then returns to operation 704 to continue to read data from the instruments.

According to one aspect of the disclosure there is thus provided a system for real time monitoring of exposure atmosphere in an aerosol exposure system. The system includes: one or more aerosol inputs; a mixing bulb; a dilution manifold with a plurality of dosimetry positions; a multiport valve; and a real-time aerosol monitor (RAM). The system is configured to: receive aerosol samples from the one or more aerosol inputs; mix the aerosol samples with humidified air using the mixing bulb; input the humidified aerosol samples into the dilution manifold. For each dosimetry position of the plurality of dosimetry positions: measure an exposure concentration for the humidified aerosol sample from the dosimetry position of the dilution manifold using the RAM; and select a next dosimetry position of the dilution manifold.

According to another aspect of the disclosure, there is thus provided a method for real time monitoring of exposure atmosphere in an aerosol exposure system. The method includes: receiving aerosol samples from one or more aerosol inputs; mixing the aerosol samples with humidified air using a mixing bulb; inputting the humidified aerosol samples into a dilution manifold. For each dosimetry position of a plurality of dosimetry positions: measuring an exposure concentration for the humidified aerosol sample from the dosimetry position of the dilution manifold using a real-time aerosol monitor (RAM); and selecting a next dosimetry position of the dilution manifold.

According to yet another aspect of the disclosure, there is thus provided a method for real time monitoring of exposure atmosphere in an aerosol exposure system. The method includes: initiating an instrumentation and establishing a connection to the instrumentation; reading data from the instrumentation at a predetermined rate; exporting the data to a user; and responsive to the user providing a user input to change a dosimetry position of the instrumentation, sending a signal to a multiport rotation valve instructing the multiport rotation valve to rotate to a desired location based on the user input.

The present disclosure is not limited in its application to the details of construction and the arrangement of components set forth in the following description or illustrated in the drawings. The examples described herein may be capable of other embodiments and of being practiced or being carried out in various ways. Also, it may be appreciated that the phraseology and terminology used herein is for the purpose of description and should not be regarded as limiting as such may be understood by one of skill in the art. Throughout the present disclosure, like reference characters may indicate like structure throughout the several views, and such structure need not be separately discussed. Furthermore, any particular feature(s) of a particular exemplary embodiment may be equally applied to any other exemplary embodiment(s) of this disclosure as suitable. In other words, features between the various exemplary embodiments described herein are interchangeable, and not exclusive.

As used in this application and in the claims, a list of items joined by the term “and/or” can mean any combination of the listed items. For example, the phrase “A, B and/or C” can mean A; B; C; A and B; A and C; B and C; or A, B and C. As used in this application and in the claims, a list of items joined by the term “at least one of” can mean any combination of the listed terms. For example, the phrases “at least one of A, B or C” can mean A; B; C; A and B; A and C; B and C; or A, B and C.

“Circuitry,” as used in any embodiment herein, may comprise, for example, singly or in any combination, hardwired circuitry, programmable circuitry such as processors comprising one or more individual instruction processing cores, state machine circuitry, and/or firmware that stores instructions executed by programmable circuitry and/or future computing circuitry including, for example, massive parallelism, analog or quantum computing, hardware embodiments of accelerators such as neural net processors and non-silicon implementations of the above. The circuitry may, collectively or individually, be embodied as circuitry that forms part of a larger system, for example, an integrated circuit (IC), system on-chip (SoC), application-specific integrated circuit (ASIC), programmable logic devices (PLD), digital signal processors (DSP), field programmable gate array (FPGA), logic gates, registers, semiconductor device, chips, microchips, chip sets, etc.

The term “coupled” as used herein refers to any connection, coupling, link, or the like by which signals carried by one system element are imparted to the “coupled” element. Such “coupled” devices, or signals and devices, are not necessarily directly connected to one another and may be separated by intermediate components or devices that may manipulate or modify such signals.

Unless otherwise stated, use of the word “substantially” may be construed to include a precise relationship, condition, arrangement, orientation, and/or other characteristic, and deviations thereof as understood by one of ordinary skill in the art, to the extent that such deviations do not materially affect the disclosed methods and systems. Throughout the entirety of the present disclosure, use of the articles “a” and/or “an” and/or “the” to modify a noun may be understood to be used for convenience and to include one, or more than one, of the modified noun, unless otherwise specifically stated. The terms “comprising”, “including” and “having” are intended to be inclusive and mean that there may be additional elements other than the listed elements.

The programs described herein are identified based upon the application for which they are implemented in a specific embodiment of the disclosure. However, it should be appreciated that any particular program nomenclature herein is used merely for convenience, and thus the disclosure should not be limited to use solely in any specific application identified and/or implied by such nomenclature.

The present disclosure may be a system, a method, and/or a computer program product. The system or computer program product may include one or more non-transitory computer readable storage media having computer readable program instructions thereon for causing a processor to carry out aspects of the present disclosure.

The one or more non-transitory computer readable storage media can be any tangible device that can retain and store instructions for use by an instruction execution device. The one or more non-transitory computer readable storage media may be, for example, but is not limited to, an electronic storage device, a magnetic storage device, an optical storage device, an electromagnetic storage device, a semiconductor storage device, or any suitable combination of the foregoing. A non-transitory computer readable storage media, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from one or more non-transitory computer readable storage media or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in one or more non-transitory computer readable storage media within the respective computing/processing device.

The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a LAN or a WAN, or the connection may be made to an external computer (for example, through the Internet using an Internet Service Provider). In some embodiments, electronic circuitry including, for example, programmable logic circuitry, Field-Programmable Gate Arrays (FPGA), or other Programmable Logic Devices (PLD) may execute the computer readable program instructions by utilizing state information of the computer readable program instructions to personalize the electronic circuitry, in order to perform aspects of the present disclosure.

It will be appreciated by those skilled in the art that any block diagrams herein represent conceptual views of illustrative circuitry embodying the principles of the disclosure. Similarly, it will be appreciated that any block diagrams, flow charts, flow diagrams, state transition diagrams, pseudocode, and the like represent various processes which may be substantially represented in computer readable medium and so executed by a computer or processor, whether or not such computer or processor is explicitly shown. Software modules, or simply modules which are implied to be software, may be represented herein as any combination of flowchart elements or other elements indicating performance of process steps and/or textual description. Such modules may be executed by hardware that is expressly or implicitly shown.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, a segment, or a portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration but are not intended to be exhaustive or limited to the embodiments disclosed. Many modifications and variations will be apparent to those of ordinary skill in the art without departing from the scope and spirit of the disclosure. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A system for real time monitoring of exposure atmosphere in an aerosol exposure system, the system comprising:
one or more aerosol inputs;
a mixing bulb;
a dilution manifold with a plurality of dosimetry positions;
a multiport valve; and
a real-time aerosol monitor (RAM), the system configured to:
receive aerosol samples from the one or more aerosol inputs;
mix the aerosol samples with humidified air using the mixing bulb;
input the humidified aerosol samples into the dilution manifold;
for each dosimetry position of the plurality of dosimetry positions:
measure an exposure concentration for the humidified aerosol sample from the dosimetry position of the dilution manifold using the RAM; and
select a next dosimetry position of the dilution manifold.

2. The system of claim 1, wherein select the next dosimetry position of the dilution manifold comprises:
automatically select the next dosimetry position at a predetermined rate.

3. The system of claim 2, wherein the next dosimetry position is selected sequentially.

4. The system of claim 2, wherein the predetermined rate is 1 Hz.

5. The system of claim 2, wherein the next dosimetry position is selected based on an input from a user.

6. The system of claim 5, wherein the user selects the next dosimetry position via a graphical user interface.

7. The system of claim 1, wherein the one or more aerosol inputs are one or more linear smoking machines.

8. The system of claim 1, wherein the plurality of dosimetry positions includes at least one filtered air control position.

9. A method for real time monitoring of exposure atmosphere in an aerosol exposure system, the method comprising:
receiving aerosol samples from one or more aerosol inputs;
mixing the aerosol samples with humidified air using a mixing bulb;
inputting the humidified aerosol samples into a dilution manifold;
for each dosimetry position of a plurality of dosimetry positions:
measuring an exposure concentration for the humidified aerosol sample from the dosimetry position of the dilution manifold using a real-time aerosol monitor (RAM); and
selecting a next dosimetry position of the dilution manifold.

10. The method of claim 9, wherein selecting a next dosimetry position of the dilution manifold comprises:
automatically selecting the next dosimetry position at a predetermined rate.

11. The method of claim 10, wherein the next dosimetry position is selected sequentially.

12. The method of claim 10, wherein the next dosimetry position is selected based on an input from a user.

13. The method of claim 12, wherein the user selects the next dosimetry position via a graphical user interface.

14. The method of claim 13, wherein:
responsive to the user selects the next dosimetry position via the graphical user interface, sending one or more commands to one or more valves of the dilution manifold to move to the next dosimetry position.

15. The method of claim 9, wherein the one or more aerosol inputs are received from one or more linear smoking machines.

16. The method of claim 9 further comprises:
initiating instrumentation and establishing connections with the instrumentation.

17. A method for real time monitoring of exposure atmosphere in an aerosol exposure system, the method comprising:
initiating an instrumentation and establishing a connection to the instrumentation;
reading data from the instrumentation at a predetermined rate;
exporting the data to a user; and
responsive to the user providing a user input to change a dosimetry position of the instrumentation, sending a signal to a multiport rotation valve instructing the multiport rotation valve to rotate to a desired location based on the user input.

18. The method of claim 17, wherein reading the data from the instrumentation at the predetermined rate further comprises:
receiving aerosol samples from one or more aerosol inputs;
mixing the aerosol samples with humidified air using a mixing bulb; and
inputting humidified aerosol samples into a dilution manifold.

19. The method of claim 17, wherein exporting the data to the user further comprises at least one of updating a user interface and exporting the data to a spreadsheet or database.

20. The method of claim 17, wherein initiating the instrumentation and establishing the connection to the instrumentation further comprises:
remotely powering the instrumentation on and connecting to the instrumentation once it has powered up.
